(19) United States
(12) Patent Application Publication
(10) Pub. No.: US 2020/0038613 A1
(43) Pub. Date: Feb. 6, 2020

(54) ORALLY-PLACED LINE SECUREMENT CLIP

(71) Applicant: CIEL MEDICAL, INC., San Carlos, CA (US)

(72) Inventors: Mary K. GARRETT, Redwood City, CA (US); Augustus C. SHANAHAN, Sunnyvale, CA (US); Kendall A. CARPENTER, Burlingame, CA (US); Dan E. AZAGURY, Palo Alto, CA (US)

(21) Appl. No.: 16/339,712

(22) PCT Filed: Oct. 16, 2017
(86) PCT No.: PCT/US2017/055632

§ 371 Date: Apr. 4, 2019

Related U.S. Application Data
(60) Provisional application No. 62/405,785, filed on Oct. 7, 2016.

(51) Int. Cl. A61M 16/04; A61B 50/20

(52) U.S. Cl. CPC A61M 16/0497; A61M 2025/0253; A61B 50/20; A61M 16/0463

(57) ABSTRACT

Described here are devices, systems, and methods for securing orally-placed tubes to the endotracheal tube of an orally-intubated patient. The device is configured to be placed against the endotracheal tube and retain one or more additional orally-placed tubes. After placing the device against the endotracheal tube, the additional tube(s) may be secured using the device.

### CROSS-REFERENCE TO RELATED APPLICATIONS

This application claims the benefit of U.S. Provisional Application No. 62/405,785, filed Oct. 7, 2016, the entirety of which is incorporated by reference herein.

### FIELD

The present invention relates to systems, devices, and methods for securing orally-placed tubes to the endotracheal tube of an orally-intubated patient.

### BACKGROUND

Patients with respiratory disease are sometimes intubated with an oral endotracheal (ET) tube and other orally-placed tubes, such as suction catheters or feeding tubes. To reduce the risk of extubation of these tubes, healthcare practitioners often secure these tubes to the patient and/or to each other. If unsecured, the additional tube(s) may become dislodged and thus prevent the patient from receiving the benefit provided by the tube(s). Presently, surgical adhesive tape is used to secure tubes to a patient's oral endotracheal tube.

### BRIEF SUMMARY

Described here are devices, systems, and methods for securing orally-placed tubes to the endotracheal tube of an orally-intubated patient. The device is configured to be placed against the endotracheal tube and to releasably secure or retain one or more additional orally-placed tubes. After placing the device against the endotracheal tube, the additional tube(s) may be secured using the device. Accordingly, there exists a need for devices and methods that allow for securement of orally-placed tubes to a conventional endotracheal (ET) tube.

In some variations, the coupling device may be a single component. In some variations, the coupling device may comprise a component that is placed against the FT tube and a component that wraps around the ET tube. In some variations, one or more components may be flexible. In some variations, a clip for the tube(s) is included on the outer face of the base. In some variations, a securing structure for a flexible component is included. In some variations, a feature to improve contact between the coupling device and the ET tube is included.

In one embodiment, a method for coupling a suction catheter to an endotracheal tube is provided, comprising attaching a suction catheter to a coupling member of a coupling device, positioning the coupling device against an endotracheal tube, surrounding a band of the coupling device around at least a portion of an outer surface of the endotracheal tube, and securing the band of the coupling device to a securing structure of the coupling device. The securing structure may be an elongate member. The method may further comprise positioning an elongate device against the coupling device or outer surface of the endotracheal tube before surrounding the band around the outer surface of the endotracheal tube. The band may comprise a first region and second region. Securing the band of the coupling device to the elongate member may comprise inserting the elongate member through an aperture in the second region of the band. The first region may comprise at least one aperture and the second region at least one aperture. The coupling member may be a clip. The method may further comprise surrounding the band of the coupling device over the clip and the suction catheter.

In another embodiment, a coupling device for a catheter and an endotracheal tube is provided, comprising a base with an outer surface and an inner surface, a securing member located on the outer face of the base, a clip protruding from the outer face of the base and spaced apart from the securing member, and a band configured to releasably couple to the securing member, wherein the base comprises an arcuate configuration with an arcuate length and a width that is orthogonal to the arcuate length, and wherein the clip is oriented to receive a tubular structure with a longitudinal axis that is generally orthogonal to the arcuate length of the base. The securing member may be an elongate member protruding from the outer surface of the base. The elongate member may have a fixed relationship with the base. The elongate member may comprise an elongate body and an enlarged distal end. The coupling elongate member may be a curved elongate member. The band may be an elastic strap. The elastic strap may have a durometer in the range of about 10 A to 90 A. The elongate member may have an orthogonal orientation relative to the outer surface of the base. The elongate member may be angled away from the clip. The elongate member may have an angle in a range of about 25 degrees to about 90 degrees to a tangential plane through a base of the elongate member, relative to the securing structure. The inner surface of the base may have a radius of curvature in the range of about 0.1″ to 2″. The base has an arc width of 0.1″ to 1.5″.

### BRIEF DESCRIPTION OF THE DRAWINGS

FIG. 1 schematically depicts an orally-intubated patient with a tube securing device.

FIGS. 2A and 2B depict perspective and side elevational views, respectively, of one embodiment of the coupling device.

FIGS. 3A to 3C depict superior perspective, side elevational and inferior perspective views, respectively, of the coupling device in FIGS. 2A and 2B, without a strap.

FIGS. 4A, 4B, 4C, 4D, and 4E depict top views of exemplary variations of the securing band described here.

FIGS. 5A, 5B, 5C, 5D, and 5E depict exemplary variations of the securing structure.

FIGS. 6A and 6B are perspective and side elevational views, respectively another exemplary embodiment of a base member.

FIGS. 7A to 7C are superior perspective, side elevational and inferior perspective views, respectively, of another exemplary embodiment of a base member.

FIGS. 8A and 8B are perspective and side elevational views, respectively, of another exemplary embodiment of a base member.

FIGS. 9A and 9B are perspective and side elevational views, respectively, of another exemplary embodiment of a base member.

FIGS. 10A and 10B are perspective and side elevational views, respectively, of another exemplary embodiment of a base member.

FIGS. 11A and 11B are perspective and side elevational views, respectively, of another exemplary embodiment of a base member.

FIGS. 12A and 12B are perspective and side elevational views, respectively, of another exemplary embodiment of a base member.

FIGS. 13A and 13B are perspective and side elevational views, respectively, of another exemplary embodiment of a base member.

FIGS. 14A and 14B are perspective and side elevational views, respectively, of another exemplary embodiment of a base member.

FIGS. 15A and 15B are perspective and side elevational views, respectively, of another exemplary embodiment of a base member.

FIGS. 16A and 16B are perspective and side elevational views, respectively, of another exemplary embodiment of a base member.

FIGS. 17A to 17C are perspective, side elevational and inferior plan views, respectively, of another exemplary embodiment of a base member.

FIG. 18 is a side cross-sectional view of an exemplary coupling device in use with an ET tube and two other tubular devices.

### DETAILED DESCRIPTION

Described here are devices, systems, and methods for coupling orally-placed tubes to an endotracheal (ET) tube or other orally-placed tube. While surgical adhesive tape is often used to secure such tubes to the ET tube and/or to the patient, adhesive tape may leave residue on the ET tube and is at risk of losing adhesive properties over time, thus increasing the potential of the securement method being unintentionally lost. Additionally, the current taping practice may also increase the risk of patient exposure to pathogens, due to surface contamination. The rolls of tape carried by healthcare personnel are rarely discarded after use on the first patient, so pathogens present in one patient may be inadvertently transferred to another patient via tape. Thus, there are advantages to having a single patient use a device for securing tubes. Also, with tubes bundled with tape, there is a risk that if one of the existing tubes is accidentally pulled, e.g. during patient transfer or from a combative or agitated patient, the entire bundle may get pulled out. Thus, there is a significant benefit to using a flexible elastic material that provides a mechanical attachment with the force of friction as opposed to an adhesive based attachment. A coupling device which couples or bundles the tubes using limited friction or mechanical force may reduce the risk of pulling out the ET tube by releasing the coupled tubes when excessive forces are applied, while reducing the risk of pulling out other tubes with it.

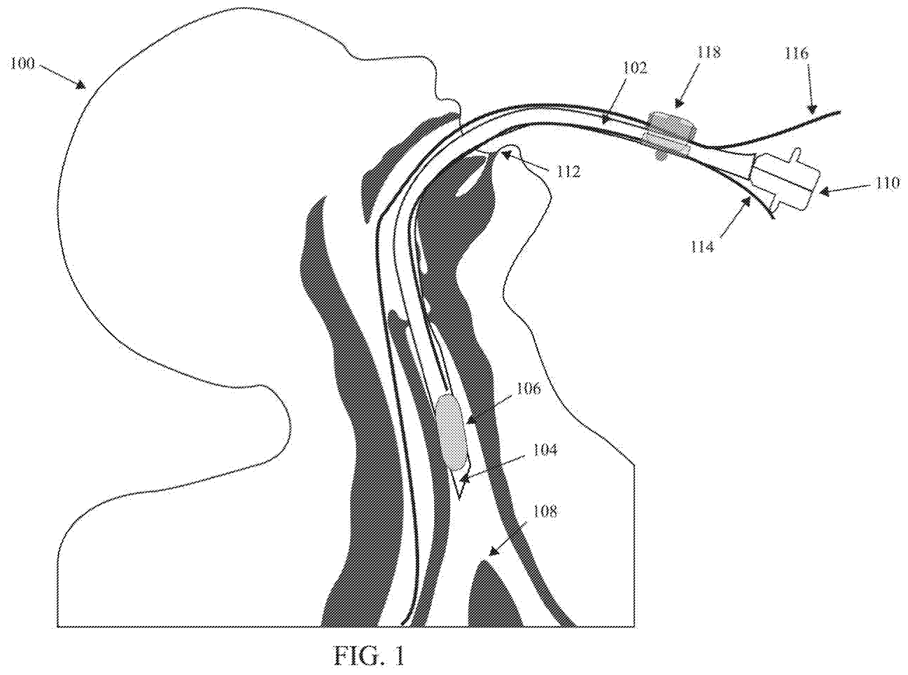

FIG. 1 is a schematic cross-sectional view of a patient 100 in which an ET tube 102 has been inserted. The distal end 104 of the ET tube 102 is positioned in the patient's trachea 106 just above the carina 108. The proximal end 110 of the ET tube 102 is protruding from the patient's mouth 112 and is connected to a ventilator (not shown). Ventilated patients may also have other orally-placed tubes, such as a suction catheter 114 and/or a feeding tube 116 which are used to insert or remove fluids from the patient's body. After these tubes 102, 114, 116 are inserted into the patient 100, some or all of the tubes 102, 114, 116 may be secured to each other, which may reduce the risk of dislodgement that may result in decreased or lost clinical function or utility. For example, the patient may chew on the tubes, and/or one or more tubes may get inadvertently pulled or pushed during patient repositioning,

FIG. 1 depicts one example of a coupling device 118 that may be used to couple the tubes 102, 114, 116 together. In this particular example, the coupling device 118 is positioned near the proximal end 110 of the ET tube 102, spaced away from the patient's mouth 112, The suction catheter 114 and/or feeding tube 116 may then be attached, retained or otherwise bundled about the ET tube 102 or coupling device 118 at the location of the coupling device 118. The coupling device 118 is then manipulated to releasably retain the suction catheter 114 and/or feeding tube 116 together with ET tube 102. In this particular example, the coupling device 118 is positioned about 3 cm to about 6 cm front the patient's mouth 112, and is not directly secured to the patient's body, but in other examples, the coupling device may be positioned closer or at the patient's mouth, and may be secured to the patient's head or per-oral location via adhesive tape, head strap or other securing mechanism. Detailed examples of various coupling devices are described below.

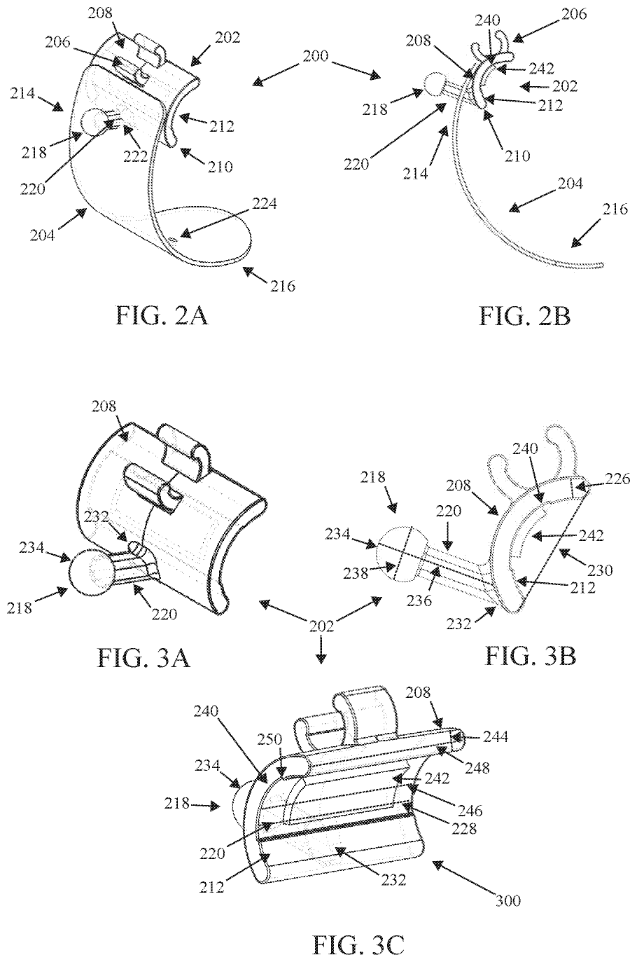

FIGS. 2A and 2B depict one example of a coupling device 200, comprising a base member 202 and a securing strap or band 204. The base member 202 may further comprise one or more coupling members 206 that are configured to retain or engage at least one orally-placed tube or elongate structure. The coupling member 206 depicted in FIGS. 2A and 2B comprises a clip that is located on an outer surface 208 of the base member 202 and configured to releasably receive a tubular structure, but in other examples may be located on an outer edge 210 or inner surface 212 of the base member 202, and/or a through lumen within the base member 202.

The securing band 204 may be configured to releasably attach the coupling device 200 to the ET tube by wrapping around or surrounding the ET tube. In other variations, the securing band 204 may be configured to additionally or alternatively retain or couple one or more orally-placed tubes or structures to the ET tube by surrounding the outer surfaces of those tubes or structures, as shown in FIG. 1. The securing band 204 may be configured to loop around, circumscribe or surround the ET tube in an orientation that is circumferential or orthogonal to the longitudinal axis of the ET tube. In other examples, however, the clip or coupling member may be configured with an orientation that is skewed or not otherwise parallel to the longitudinal length or axis of the base member.

Referring back to FIGS. 2A and 2B, the securing band 204 may comprise a flexible or elastic body, with a first end section 214 and a second end section 216. The securing band 204 is configured such that the first end section 214 may be releasably coupled to the base member 202 to a securing member 218 of the base member 202. The attachment of the first end section 214 to the securing member 218 may be performed at the point-of-manufacture, or at the point-of-use. The securing member 218 may comprise an elongate body 220 which is configured to be releasably inserted through one or more apertures 222 located on the first end section 214 of the band 204. In use, after the first end section 214 is attached or pre-attached to the securing member 218, and the base member 202 is placed against the ET tube or other tubular structure, and the other catheters or tubes are attached or bundled to the ET tube or base member 202, the band 204 is then wrapped around the ET tube and other tubes, and then the free second end section 216 of the band is secured to the base member 202 or to itself, and enclosing the ET tube and the other tubes.

In this particular embodiment, the second end section 216 comprises a second aperture 224 which is configured to be releasably coupled to the same securing member 218 as the aperture 222 of the first end section 214, after the band 204 has been wrapped around the ET tube and back to the base member 202. In other examples, however, a separate or additional securing member may be provided on the base member to secure the second end section.

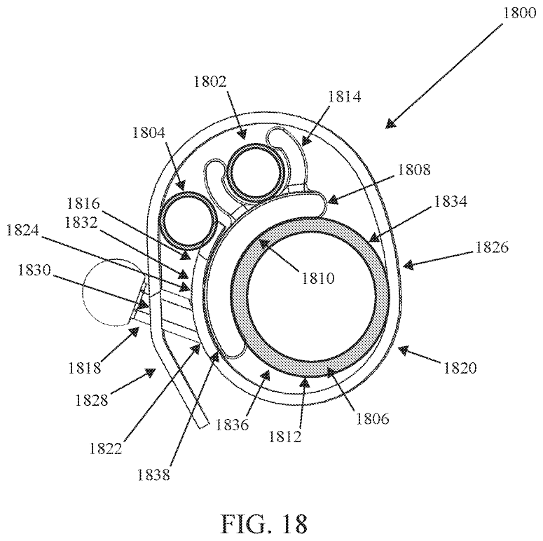

FIG. 18 depicts one example of a coupling device 1800 used to couple a suction catheter 1802, feeding tube 1804 and an ET tube 1806. The coupling device 1800 comprises an arcuate base member 1808, where the concave inner surface 1810 of the base member 1808 has been placed in contact against a convex outer surface 1812 of the ET tube 1806. The base member 1808 comprises a clip 1814 projecting from a convex outer surface 1838 of the base member 1808 into which a first tubular structure, such as a suction catheter 1802, has been releasably attached in a longitudinal orientation that is generally parallel to the longitudinal orientation of the ET tube. A second tubular structure, such as a feeding tube 1804, has been placed against the outer surface 1838 of the base member 1808, in a middle region 1816 between the elongate securing member 1818 and the clip 1814, in an orientation generally parallel to the ET tube 1806 and the suction catheter 1802. The securing band 1820 is coupled to the securing member 1818 via a first aperture 1822 in the first end region 1824 of the band 1820. The band 1820 initially wraps around the ET tube 1806 in a direction away from the clip 1814, and then back towards the clip 1814. The middle section 1826 of the band 1820 covers the clip 1814 and the suction catheter 1802, and the feeding tube 1804. The second end region 1828 is then attached to the same elongate securing member 1818 by a second aperture 1830 in the second end region 1828.

Although the feeding tube 1804 is secured at a location 1832 between the securing member 1818 and the clip 1814, and between the band 1820 and the base member 1808, the feeding tube 1804 or other tubes may also be positioned between the band 1820 and the ET tube 1806, without the base member 1808. For example, a tube or elongate structure may be secured at locations 1834, 1836 to the side of clip 1814 and base member 1808.

Referring back to the embodiment depicted in FIGS. 2A and 2B and in FIGS. 3A to 3C, the base member 202 may comprise a generally flat or layer structure, that may have a planar, non-planar, or curved configuration, and may be rigid, flexible or elastic material, or combination thereof. In the exemplary base member 202 depicted in FIGS. 3A to 3C, the base member 202 comprises a thickness or height 226 that is substantially lower than the longitudinal length 228 or arcuate width 230 of the base member 202. The arcuate configuration of the base member 202 or at least the inner surface 212 of the base member 202 is provided along the width 230 so that the coupling device 200 is configured to be seated or positioned against the outer surface of an ET tube so the that coupling member 206 is configured to receive a tube or elongate structure with a longitudinal axis that is generally parallel to the longitudinal axis of the ET tube. In some variations, the inner surface of the base member may be arcuate or concave curved, but the outer surface of the base member may be flat, concave, or have some other non-complementary shape relative to the inner surface of the base member.

As illustrated in FIGS. 3A and 3B, the securing member 218 of the base member 202 may comprise an elongate body 220 that projects from the outer surface 208 of the base member 202. The elongate body may comprise a base 232 attached to the outer surface 208, a distal end 234, and a longitudinal axis 236, wherein the distal end 234 may have a larger cross sectional area or larger maximum transverse dimension 238 relative to the rest of the elongate body 220 or the longitudinal axis 236. In this embodiment, the elongate body 220 has a straight configuration with an orientation that is about 90 degrees or orthogonal with respect to the longitudinal length 228 of the base member 202, but forms an acute angle along the width 230 of the base member 202, being angled toward the coupling member 206.

Referring back to FIG. 18, the coupling device 1800 may comprise a smooth concave inner surface 1810, but in other examples, the inner surface of the base member may comprise texturing, ridges, projections, recesses, and combinations thereof. In some further examples, the inner surface may also comprise an adhesive layer configured to adhere to the outer surface of an ET tube. For example, the coupling device 200 depicted in FIGS. 2B, 3B and 3C, comprises an inner surface 212 with a recess 240 and a protruding structure 242 within the recess 240. In this particular example, the recess 240 has lateral edges 250 that are spaced apart from the lateral edges 244 of the base member 202, while the longitudinal edges 246 of the recess 240 spans across both longitudinal edges 248 of the base member 202. In other examples, multiple recesses may be provided, with or without protrusions therein, or multiple protrusions may be provided, with or without recesses. The recesses and protrusions may have any of a variety of shapes, including rectangles, squares, triangles, ovals, polygons, pyramid, rounded bumps, etc. and the recesses and protrusions may have a similar or dissimilar shape.

In some examples, the inner surface 212 and/or the protrusions 242 comprise a material that provides increased tackiness or grip that may reduce sliding relative to the ET tube. The coupling device 300 in FIG. 3C, for example, may comprise a piece of tacky, elastomeric material, such as a styrenic block copolymer or silicone, for example. In still other embodiments, a surface texture may be provided onto the inner surface, such as a bead blasted surface finish or a rough, sand-paper like grit finish.

In the exemplary embodiments described above or described below, the longitudinal length of the base member of the coupling device may be about 0.75″ or may be in the range of 0.1″ to 2″, or a range of 0.4″ to 1″. The linear width of the base member of the coupling device may be 0.47″, or can be in the range of 0.1″ to 2″ or 0.25″ to 0.75″. The radius of curvature of the base of the coupling device is 0.2″ and can range from 0.1″ to 2″ or 0.1″ to 0.5″, or may in some instances be flat and have no curvature. The arc width of the base member of the coupling device may be 0.62″ and can range from 0.1″ to 1.5″, or from 0.4″ to about 1″.

In some variations, the endotracheal tubes that the coupling device may be secured to can range from 3 mm to 15 mm in outside diameter, with typical adult endotracheal tubes in the range of about 9 mm to 12 mm in outside diameter. The tubing or elongate devices that may be attached to the ET tube using the coupling device may include, but are not limited, to sump pump tubes, suction catheters, nasogastric feeding tubes, esophageal monitoring catheters and cuff pressure monitoring catheters, and the like. The coupling members of the coupling devices may be configured to receive devices or structures in the range of about 14 Fr to about 18 Fr, or about 8 Fr to 18 Fr. Larger structures and/or structure with atypical shapes may be retained in other locations, such as locations 1832, 1834, 1836 as depicted in FIG. 18, without using the coupling member of the base member.

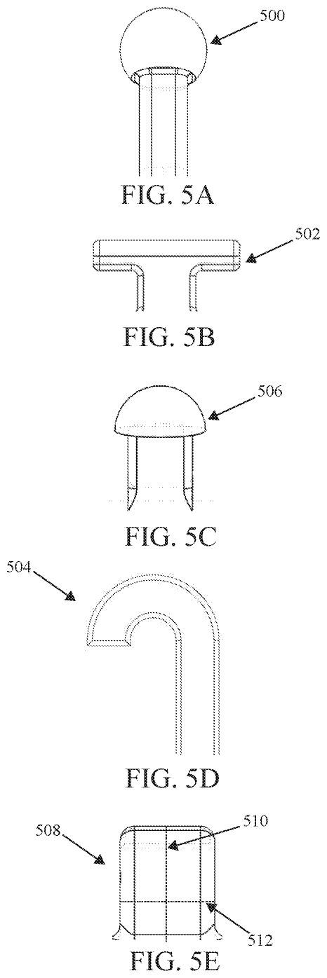

The enlarged distal end of the elongate body, if provided, may comprise a curved configuration, a spherical or oblong shape, pyramidal shape, cuboidal shape, or other shape. The cross-section of the elongate body is shaped as a square with rounded edges, though may be any shape in cross section, such as a circle, square or rectangle. Exemplary variations of the elongate body distal end are depicted in FIGS. SA to SD, which include a spherical shape 500, a T-bar shape 502, a hook shape 504, a hemispherical or mushroom shape 506. FIG. 5E also depicts another example of a securing member 508, wherein securing member 508 does not have an enlarged distal end or narrow proximal neck. In this variation, the securing member 508 has a generally uniform cross-sectional shape and size along its longitudinal length or height. The length of the securing member 508 may be similar to other securing members described herein, but in other variations, the vertical height 510 of the securing member 508 may be in the range of about 50% to 200% of its width 512. In still other variations, the height 510 may be in the range of about 75% to 150%, or about 100% to 150%.

The elongate body of the securing member may have a length of about 0.3″ but in other examples, may have a length in the range about 0.01″ to 1″, or 0.2″ to 0.8″. The enlarged distal end may have a cross-sectional area (transverse to the longitudinal axis of the elongate body) of about 0.085 in² but in other examples may be in the range of 0.01 in² to 2 in², or 0.5 in² to 1.2 in². The maximum transverse dimension of the distal end may be about 0.17″, but may be in the range of about 0.1″ to 1″, about 0.15″ to about 0.8″, or about 0.1″ to about 0.5″. The elongate body may be at an angle 45° to the tangential plane or surface of the outer surface of the base, angled toward the clip or securing member, but in other examples, the elongate body may have an angle in the range of about 10° to 270°, 30° to 135′, or about 45° to 90°. The maximum transverse dimension of the distal end relative to the maximum transverse dimension of the elongate body (with respect to the longitudinal axis) may be about 30% larger, and in other embodiments, may be in the range of about 10% to 100% larger, about 20% to about 200% larger, or about 30% to about 250% larger.

The clip protruding from the outer face of the base is 0.21″ from the elongate member along the arcuate and can range from 0.0″ to 2.000″. The clip protruding from the outer face of the base is 45° from the elongate member and can range from 10° to 180°. The wings of the clip protruding from the outer face of the base are spaced 0.15″ apart at the opening and can range from 0.05″ to 0.5″. The interwidth or interspacing of the clip may be configured so that it is large enough that it does not significantly collapse the tube it is securing while providing some friction or mechanical interfit with the tubing or elongate structure. In other embodiments, however, such as the securing member 908 of base member 900 in FIGS. 9A and 9B, or the second securing member 1104 of base member 1100 in FIGS. 11A and 11B, the securing member may restrict or resist certain movements of tubes or structures placed against or within them, but without necessarily providing a friction fit.

The clip may be configured to protrude from the outer surface of the base member to a height of approximately 0.16″, but in other variations, the protrusion length may be in the range of about 0.1″ to 0.5″. The height of the clip may be selected to reduce the risk of partial or complete occlusion of soft tubes that are captured within or inserted into the clip. Some orally placed catheters, such as suction catheters, may be soft and collapsible.

The clip(s) may be configured for use with a range of tubes that are commonly used with intubated patients, which include but are not limited to devices such as sump pump tubes, suction catheters, nasogastric feeding tubes, esophageal monitoring catheters and cuff pressure monitoring catheters, which are typically between 8 Fr to 18 Fr, and more commonly in the range of about 14 Fr to 18 Fr.

The securing bands described herein may have a width of about 0.6″, or have a width in the range of about 0.1″ to 2″, or about 0.25″ to 1″. The length may be about 2.5″, or in the range of about 0.5″ to 9″, or about 1.5″ to 4″, for example. The securing band may be made of a flexible and/or elastic material, and in some further variations, may comprise a material that has a high coefficient of friction. Specifically, it may be made of a clear, food-grade silicone of 50 A durometer with a thickness of 0.030″. However the durometer may be anywhere from 10 A to 90 A durometer and the thickness may be anywhere from 0.005″ to 0.15″ or 0.020″ to 0.060″. The coefficient of friction may be anywhere from 0.25 to 0.9 and may be provided by the innate material properties of the securing band material and/or a texture on the securing band. The securing band may comprise a material such as silicone or other synthetic rubbers, a thermoplastic elastomer, a polyurethane, a natural rubber, or latex, for example.

In the particular embodiment depicted in FIGS. 2A and 2B, the distance between the two apertures on the securing band 204 is 1.5″, but in other examples, the distance may be in the range of about 0.25″ to 8″ or about 1.2″ to 2″. The apertures 222, 224 in are shaped as circles, but in other examples may be square, oval or slit shaped. In FIGS. 2A and 2B, one aperture 222 is about 0.03″ in diameter and the other aperture 224 is about 0.060″ in diameter. In other examples, the apertures may have the same or different size and/or shape, and each aperture may have a size or maximum transverse dimension in the range of about 0.005″ to 0.25″, or more.

Although the exemplary coupling device 200 comprises a separate base member 202 and band 204, in other variations, the base member and band may be integrally formed or permanently attached at the first end of the hand. Providing separate components, however, may facilitate the use of different materials for each component, e.g. providing a rigid base member with the flexible securing band. In other variations, the securing member may be separate from the base member and is configured and used to secure different apertures of the band together when the apertures are placed in an overlapping configuration or are sufficiently adjacent to each other to permit securing by the securing member. A separate securing member may be, for example, a double button or double disc design, or a double T-bar design, or a combination thereof. In still other variations, the base member and band may be molded with two different materials, either through a two shot mold or an overmold process, allowing the use of different materials for each the base and the band while still creating the assembly as a single component.

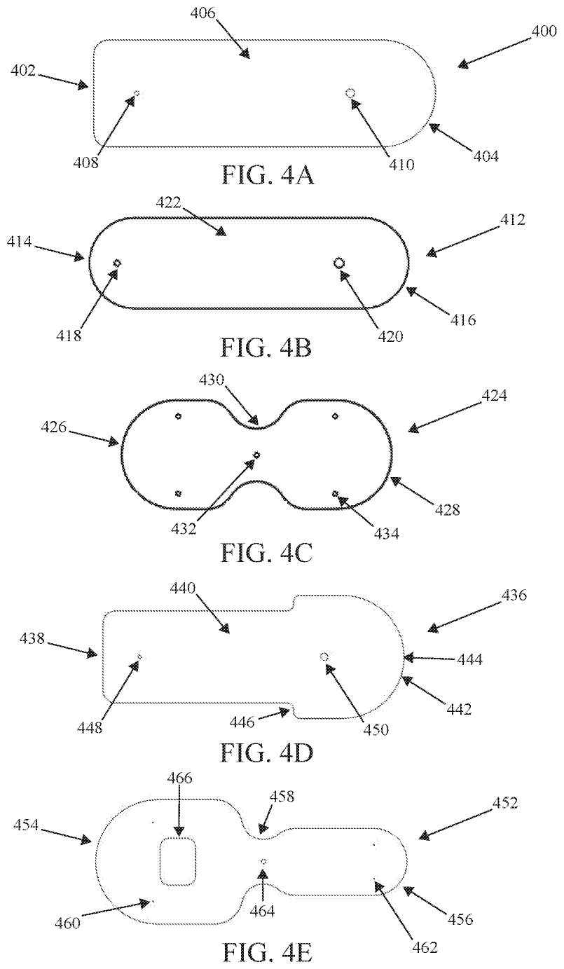

The securing band of the coupling devices may comprise a variety of configurations. For example, FIG. 4A depicts a band 400 with a squared first end section 402 and a semi-circular second section 404 and a middle section 406 with a generally uniform width. The first end section 402 and second end section 404 comprise each comprise a single centrally positioned aperture 408, 410, but with different sizes. In other examples, multiple apertures may be provided, to provide greater flexibility in device use with a greater variety of components.

FIG. 4B depicts another variation, comprising a securing band 412 with semi-circular first and second end sections 414, 416, and also with centrally positioned, different size apertures 418, 420, that are also more closely spaced to the distalmost edges of their respective sections. The middle section 422 comprises a uniform with the diameters of the first and second end sections 414, 416.

FIG. 4C depicts a securing band 424 with semi-circular first and second end sections 426, 428, but with a shorter length middle section 430 that also has a reduced width compared to the diameter or maximum widths of the first and second end sections 426, 428. A centrally positioned aperture 432 is also provided in the middle section 430, while each of the first and second end sections 426, 428 comprise a pair of laterally positioned apertures 434 of uniform size.

FIG. 4D depicts a securing band 436 comprising a first end section 438 comprising a squared configuration with rounded corners, a middle section 440 with the same width as the first end section 438, and a second end section 442 comprising a mushroom or bulbous configuration, comprising a semi-circular region 444 and polygonal region 446, wherein the diameter of maximum width of the section end section 442 is greater than the width of the first end section 438 or the middle section 440. Centrally positioned smaller and larger apertures 448, 450 are provided in the respective end sections 438, 442.

FIG. 4E illustrates still another variation of a securing band 452, comprising a wider or large oblong first end section 454, a smaller oblong second end section 456 and a narrowed middle section 458 that comprises a narrower minimum width than the maximum widths of both the first and second end sections 454, 456. The first end section 454 comprises two lateral apertures 460 and the second end section comprises two lateral apertures 462 with a similar size to the apertures 460 of the first section. The middle section also comprises a centrally positioned aperture 464, which may be larger than apertures 460, 462. In addition, securing band 452 comprises a window 466, which is located in the first end section 454, but in other variations, may be located in the middle section and/or second end section. The window 466 may be used to provide a provide visibility to a feature of the ET tube or other tubes and structures coupled to the coupling device. In other examples, the coupling device may comprise a base member with a coupling member and/or securing member that is configured to protrude from the window 466.

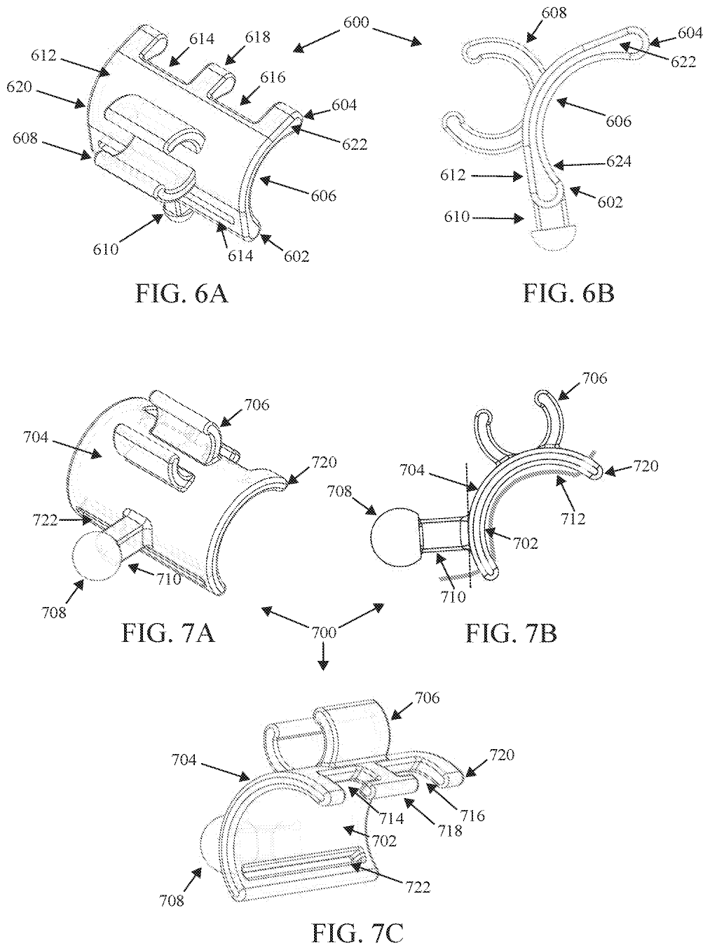

Likewise, the base members of the coupling devices may also comprise a variety of features that may be mixed and matched, examples of which are provided below. In FIGS. 6A and 6B, for example, the base member 600 comprises a variable thickness or height, with a greater thickness at its lateral edges 602, 604 in comparison to its central region 606. In contrast to the lateral location of the coupling member 206 of base member 202 in FIGS. 2A and 2B, the coupling member 608 of base member 600 is located centrally relative to the lateral edges 602, 604. In addition, the securing member 610 extends laterally from one of the lateral edges 602 of the base member, rather than extending from the outer surface 612 of the base member 600. The base member further comprises an opening or slot 614, through which a securing band may be passed. In this example, the lateral edge 604 opposite the lateral edge 602 with the securing member 610, comprises two recessed regions 614, 616 which are separated by an edge projection 618. In this specific example, the edge projection 618 extends laterally to the same lateral distance as the lateral edges 604 of the base member 600 and has the same orientation, but in other examples, the edge projection may extend more or less than the lateral edges 604, and/or may be angled toward either of the distal edges 620, 622 or angled toward the outer surface 612 or the inner surface 624 of the base member 600. The edge projection 618 may also be used to retain or couple to a region or structure of a securing band. The base member 600 comprises a concave curve along the lateral dimension or axis on its inner surface 624 and a convex curve along the lateral dimension or axis on its outer surface 612, without any curvature along the longitudinal dimension or axis.

FIGS. 7A to 7C depicts another variation of a base member 700, comprising curved inner and outer surfaces 702, 704, and laterally positioned coupling member 706 and securing member 708. In this particular embodiment, the securing member 708 comprises an elongate body 710 that has an orthogonal or 90° orientation in the lateral and longitudinal directions, relative to the tangential plane between the elongate body 710 and the outer surface 7045 of the base member 700. Like the base member 600 in FIGS. 6A and 6B, a slot 722 is provided to receive a securing band 712, as shown in FIG. 7B, but here the two lateral recessed regions 714, 716 flank a T-bar projection 718 to retain the securing band 712. The T-bar projection 718 is also aligned with the lateral edge 720 of the base member 700, but in other variations, the T-bar may have radial orientation rather than the longitudinal orientation shown in FIG. 7C, and may also project a lesser or greater distance, or be angled in a radial direction

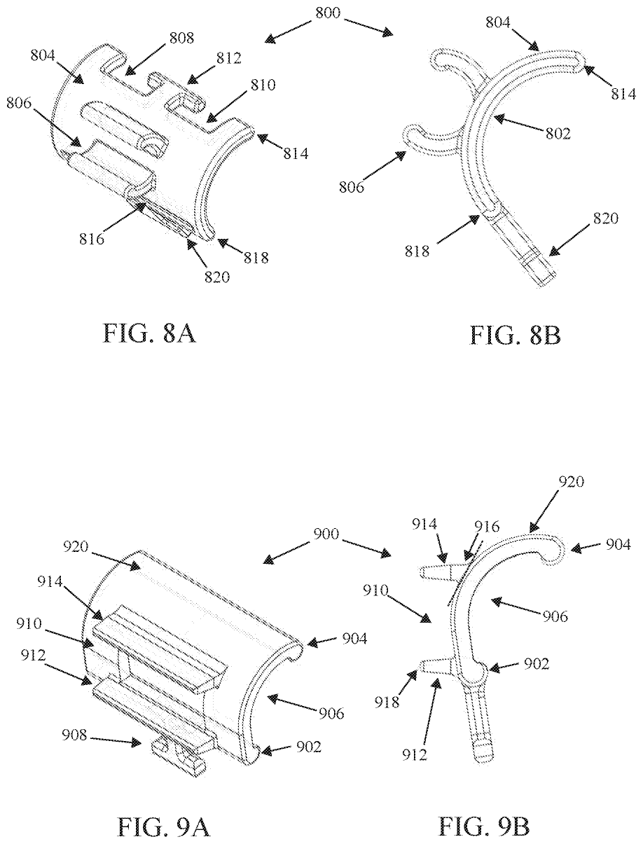

FIGS. 8A and 8B depict a base member 800 with curved inner and outer surfaces 802, 804 and a centrally located clip or coupling member 806, as well as two lateral recesses 808, 810 and a T-bar projection 812 along one of its lateral edges 814 to receive a securing band along with a slot 816. Adjacent to the slot 816, however, instead of a linear elongate body projecting from the other lateral edge 818, a second T-bar projection 820 is provided. This projection 820 is longer and wider than the first projection 812, but in other examples, the projections may have a similar configuration.

FIGS. 9A and 9B depict a curved base member 900 with a variable thickness that is greater at its lateral edges 902, 904 than its central region 906. A T-bar projection 908 extends from one of the lateral edges 902, but not the other lateral edge 904. In addition, the coupling member 910 comprises straight struts 912, 914, increase of the angled or arcuate struts provided in other embodiments herein. In this example, the straight struts taper from their base 916 to their distal tip 918, and generally share the same orientation even though each is positioned on different portions of the curved outer surface 920, with straight strut 912 being orthogonal to the lateral T-bar projection 908, and the other straight strut 914 having the same orientation as straight strut 912, but is at a 45° angle relative to the tangent of its location on the curved outer surface 920 of the base member 900. In some examples, straight struts may be provided to restrict circumferential displacement of any tubes or structures placed between the straight struts, but without necessarily generating any pinching or clipping forces against the tube or structure. Retention of the tube or structure may then be provided in conjunction with a securing band that is wrapped over the straight struts.

In the embodiments described above, the inner surface of the base member generally encompass less than 180° of the radius of curvature of the inner surface, and cannot independently attach or couple to an ET tube, without a securing band or other structure to further wrap or circumscribe the ET tube.

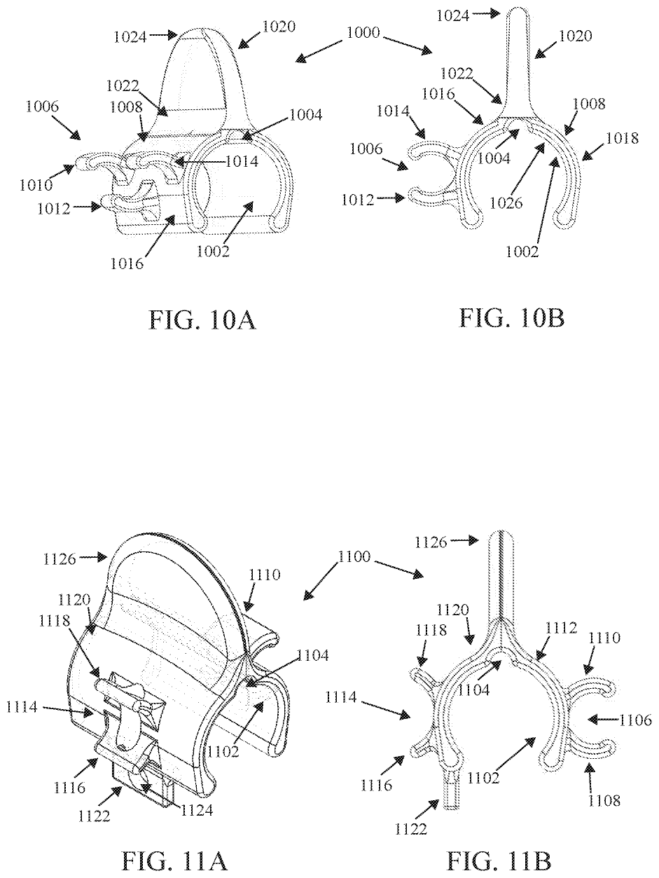

In FIGS. 10A and 10B, however, the base member 1000 comprises an inner surface 1002 that circumscribes more than 180° degrees of its radius of curvature, and generally about 270°, or in the range of about 240° to about 330°, or about 260° to about 280°. This configuration may permit the base member 1000 to attach or couple to an ET tube or other structure without requiring a securing band. A longitudinally oriented recess 1004 may be provided on the inner surface 1002 to retain a tube between the ET tube and the inner surface 1002. In addition, one or more coupling members 1006 may be provided on the outer surface 1008 of the base member 1000. The coupling members may be configured with the aligned arcuate struts found in several embodiments described herein, but in the embodiment depicted in FIGS. 10A and 10B, the coupling member 1006 comprises three offset coupling struts 1010, 1012, 1014, instead of two aligned struts, to releasable, receive a tubular member. Although a single coupling member 1006 is provided on one lateral edge 1016 of the base member 1000, in other examples, another coupling member may be provided on the other lateral edge 1018. Also, in other variations, the coupling member may comprise four or more offset struts, rather than three offset struts. A midline handle or grasping member 1020 is also provided on the outer surface 1002. This handle 1020 has a semi-circular configuration and a longitudinal length than spans both longitudinal edges of the base member 1000, and has a slight tapering from its base 1022 to its distal tip 1024. In other examples, the handle may have a different configuration. For example, the handle may be rectangular or square, may be located at or closer to one of the lateral edges, or may span less than, or extend past, one or more longitudinal edges, and/or may be angled longitudinally and/or laterally. In sonic variations, as shown in FIG. 10B, the lateral edges 1016, 1018 may comprise a greater thickness than the central region 1026 of the base member 1000, which may further facilitate its ability to attach or couple to an ET tube.

FIGS. 11A and 11B depict another example of a base member 1100 with an inner surface 1102 that encompasses more than 180° of the inner surface 1102 relative to its radius of curvature, and may be configured to attach independently to an ET tube. In this example, an inner surface recess 1104 is provided to retain a tubular structure like in the base member 1000 in FIGS. 10A and 10B. A coupling member 1106, comprising a pair of aligned clip struts 1108, 1110, is provided adjacent to one lateral edge 1112, as well as a second coupling member 1114, comprising a pair of aligned arcuate struts 1116, 1118 is provided adjacent to the other lateral edge 1120. In this embodiment, the first coupling member 1106 is configured to encompass more than 180° of its radius of curvature, while the second coupling member 1114 encompasses less than 180° of its radius of curvature. The second coupling member 1114 also has a greater radius of curvature than the first coupling member 1106. An edge tab 1122 with an aperture 1124 is provided on the lateral edge 1120. This aperture 1124 may be used to secure the base member 1100 to another structure or anatomical region of the patient, and/or may be used to aid in retaining a tubular structure against the second coupling member 1114. A centrally located handle 1126 is also provided in this exemplary embodiment.

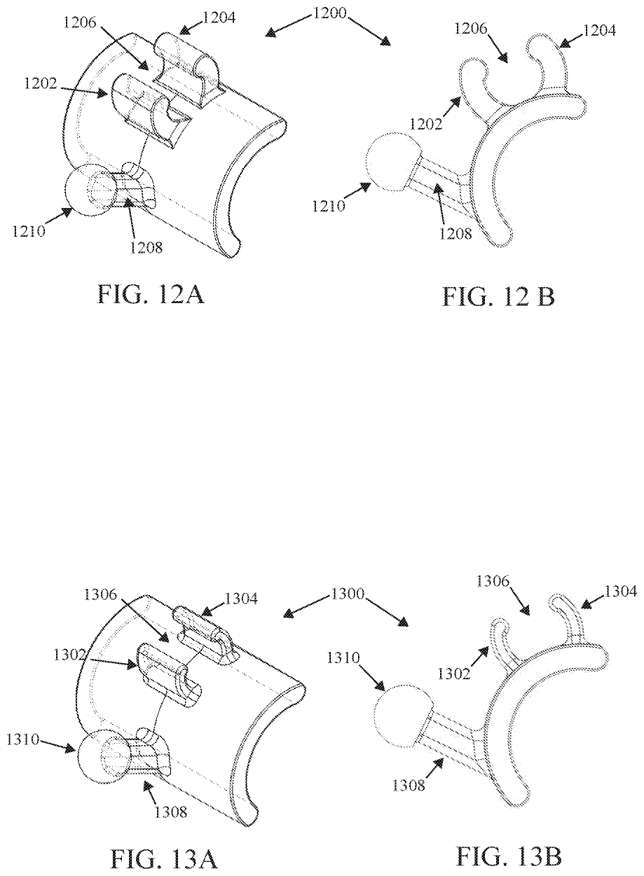

FIGS. 12A and 12B depict a variation of a base member 1200, wherein the curved struts 1202, 1204 of the coupling member 1206 are thicker and/or spaced more closely together. The spacing between the coupling member 1206 and the elongate body 1208 of the securing member 1210 is also closer together, and at the lower end of the ranges specified above.

FIGS. 13A and 13B depict a variation of a base member 1300, wherein the curved struts 1302, 1304 of the coupling member 1306 are thinner and/or spaced farther apart, compared to other embodiments described herein. The elongate body 1308 is also thicker than other embodiments described herein, and the enlarged distal end 1310 of the elongate body 1308 comprises a greater percentage of a sphere, compared to other examples. Whereas the hemispherical embodiments may comprise about 40 to 60% of a sphere, in this example, the distal end 1310 may be in the range of about 60% to about 90%, or about 75% to about 90% of a sphere.

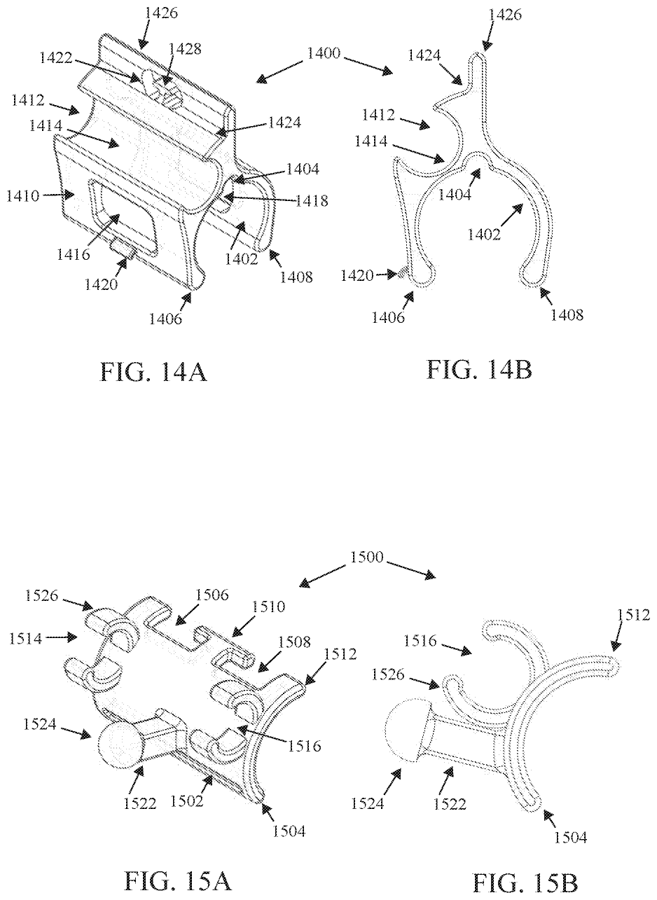

FIGS. 14A and 14B depict another variation of a base member 1400 wherein the inner surface 1402 encompasses more than 180° of its radius of curvature and comprises a tubular longitudinal inner recess 1404 and thickened lateral edges 1406, 1408, Instead of a coupling member that projects from the outer surface 1410 of the base member 1400, however, the coupling member 1412 comprises a longitudinal outer recess 1414 configured to releasably receive a tubular structure. The outer recess 1414 may or may not be configured with a convex surface that encompasses more than 180° of its radius of curvature and configured to independently couple to the tubular structure. Additionally, the base member 1400 includes lateral windows 1416, 1418 which may aid in viewing certain features of the ET tube or the contents therein. A curved tab 1420 is also provided between the lateral edge 1420 of the base member and the window or longitudinal recess 1414. This curved tab 1420 may be used in conjunction with the circumscribed, recessed T-bar projection 1428 located within a third window 1422 located within a midline flange 1424 projecting from the midline outer surface 1426 of the base member 1400, to retain any structure positioned in the outer recess 1414, or other retention function as described herein.

FIGS. 15A and 15B depict still another variation of a base member 1500, comprising a slot 1502 adjacent to one lateral edge 1504 for receiving a securing band and two recesses 1506, 1508 and a T-bar projection 1510 along the other longitudinal edge 1512. In this particular variation, two coupling members 1514, 1516 are provided, wherein the coupling member 1514, 1516 are located on opposing longitudinal edges 1518, 1520 but are laterally aligned to facilitate attachment of both coupling members 1514, 1516 to the same tubular structure. As illustrated best in FIG. 15B, this configuration of the coupling members 1514, 1516 may permit the coupling of a tubular structure more closely next to the elongate body 1522 of the securing member 1524, while possibly avoiding interference from the arcuate struts 1526 of the coupling members 1514, 1516.

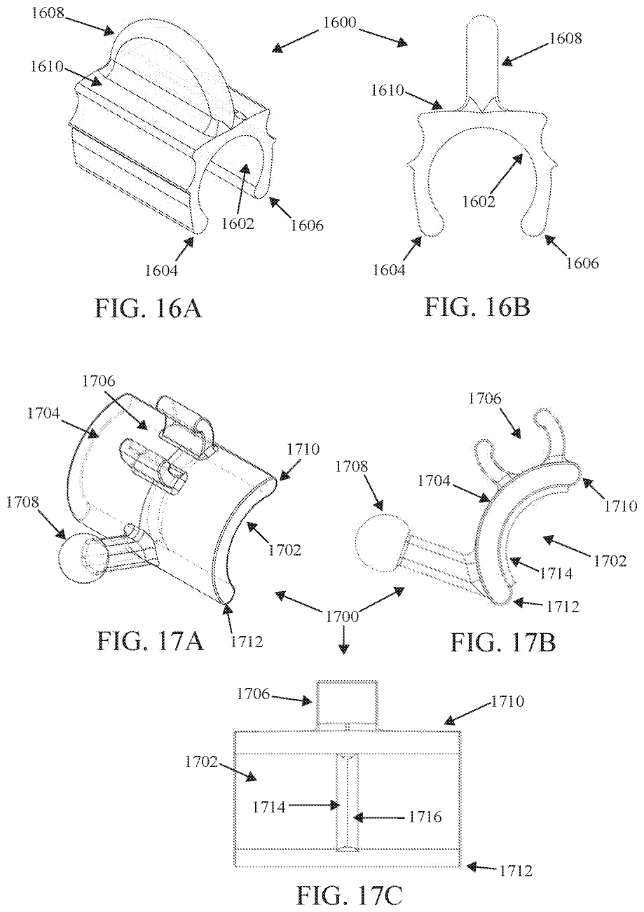

FIGS. 16A and 16B depicts another variation of a base member 1600, comprising an inner surface 1602 encompassing greater than 180° of its radius of curvature and comprising thickened lateral edges 1604, 1606. A midline semi-circular handle 1608 is also provided on its outer surface 1610. In addition, shallow longitudinal recesses are provided adjacent to each side of the handle 1608.

In the final example, illustrated in FIGS. 17A to 17C, the base member 1700 comprises curved inner and outer surfaces 1702, 1704, respectively, along with a coupling member 1706 and an angled securing member 1708 located on the outer surface 1704 adjacent their respective lateral edges 1710, 1712, In addition, the inner surface 1702 comprises two flat but raised structures 1714, 1716. These structures 1714, 1716 may be configured with similar materials) and/or similar function(s) as the protrusion 242 described for coupling device 200.

Although the embodiments herein have been described in relation to certain examples, various additional embodiments and alterations to the described examples are contemplated within the scope of the invention. For example, one or more features of the coupling devices, base members, coupling members, securing member associated with embodiments 200, 600, 700, 800, 900, 1000, 1100, 1200, 1300, 1400, 1500, 1600 and 1700 are contemplated be added or substituted to other embodiments, and/or deleted from each embodiment, or used or substituted with any of the elongate body configurations and securing bands described herein. Thus, no part of the foregoing description should be interpreted to limit the scope of the invention as set forth in the following claims. For all of the embodiments described above, the steps of the methods need not be performed sequentially. Accordingly, it is not intended that the invention be limited, except as by the appended claims.

1. A coupling device for a catheter and an endotracheal tube, comprising:
a base with an outer surface and an inner surface;
a securing member located on the outer surface of the base;
a clip protruding from the outer surface of the base and spaced apart from the securing member; and
a band configured to releasably couple to the securing member;
wherein the base comprises an arcuate configuration with an arcuate length and a width that is orthogonal to the arcuate length; and
wherein the clip is oriented to receive a tubular structure with a longitudinal axis that is generally orthogonal to the arcuate length of the base.

2. The coupling device of claim 1, wherein the securing member is an elongate member protruding from the outer surface of the base.

3. The coupling device of claim 2, wherein the elongate member has a fixed relationship with the base.

4. The coupling device of claim 3, wherein the elongate member comprises an elongate body and an enlarged distal end.

5. The coupling device of claim 2, wherein the elongate member is a curved elongate member.

6. The coupling device of claim 1, wherein the band is an elastic strap.

7. The coupling device of claim 6, wherein the elastic strap has a durometer in a range of about 10 A to 90 A.

8. The coupling device of claim 3, wherein the elongate member has an orthogonal orientation relative to the outer surface of the base.

9. The coupling device of claim 3, wherein the elongate member is angled away from the clip.

10. The coupling device of claim 9, wherein the elongate member has an angle in a range of about 25 degrees to about 90 degrees to a tangential plane through a base of the elongate member, relative to the securing member.

11. The coupling device of claim 1, wherein the inner surface of the base has a radius of curvature in a range of about 0.1″ to about 2″.

12. The coupling device of claim 11, wherein the base has an arc width of about 0.1″ to about 1.5″.

13. A method for coupling a suction catheter to an endotracheal tube, comprising:
attaching a suction catheter to a coupling member of a coupling device;
positioning the coupling device against an endotracheal tube;
surrounding a band of the coupling device around at least a portion of an outer surface of the endotracheal tube; and
securing the band of the coupling device to a securing structure of the coupling device.

14. The method of claim 13, wherein the securing structure is an elongate member.

15. The method of claim 14, further comprising positioning an elongate device against the coupling device or outer surface of the endotracheal tube before surrounding the band around the outer surface of the endotracheal tube.

16. The method of claim 15, wherein the band comprises a first region and second region.

17. The method of claim 16, wherein securing the band of the coupling device to the elongate member comprises inserting the elongate member through an aperture in the second region of the band.

18. The method of claim 17, wherein the first region comprises at least one aperture and the second region at least one aperture.

19. The method of claim 13, wherein the coupling member is a clip.

20. The method of claim 19, further comprising surrounding the band of the coupling device over the clip and the suction catheter.
